(19) United States
(12) Patent Application Publication
(10) Pub. No.: US 2026/0294839 A1
(43) Pub. Date: Oct. 1, 2026

(54) COMPOSITION AND METHOD OF MAKING

(71) Applicant: AGP Innovation Limited, Castle Donington (GB)

(72) Inventor: Andrew THAIN, Castle Donington (GB)

(21) Appl. No.: 19/479,270

(22) PCT Filed: Apr. 26, 2024
(86) PCT No.: PCT/EP2024/061625

§ 371 Date: Oct. 27, 2025

(30) Foreign Application Priority Data
Apr. 28, 2023 (GB) 2306332.4

(51) Int. Cl. A61K 31/047; A01N 31/02; A01N 35/02; A01P 1/00; A01P 3/00; A61K 8/34; A61K 8/35; A61K 31/12; A61K 33/40; A61P 31/04

(52) U.S. Cl. CPC A61K 31/047; A01N 31/02; A01N 35/02; A01P 1/00; A01P 3/00; A61K 8/345; A61K 8/35; A61K 31/12; A61K 33/40; A61P 31/04

(57) ABSTRACT

An antimicrobial composition comprising glycerol and dihydroxyacetone is disclosed. Also, a method of making the antimicrobial composition comprising mixing a reagent comprising glycerol and an oxidant, optionally with a catalyst, is disclosed. The composition can be included in products such as a wound dressing, a food product, a cosmetic product, and a cleaning product. The compositions can be used in the prevention or treatment of a skin condition and in therapy.

### TECHNICAL FIELD

The present invention relates to an antimicrobial composition and a method of making said composition.

### BACKGROUND

Manuka honey originates from Australia and New Zealand and is made by bees that forage on the Manuka bush (Leptospermum scoparium). It has been used in medicine and natural remedies for many years due to its anti-microbial properties. These properties originate from a compound called methylglyoxal (MGO). MGO concentration is much higher in Manuka honey than in other forms of honey. MGO is formed naturally from dihydroxyacetone (DHA), another compound present in Manuka honey.

There are drawbacks to the use of Manuka honey. Due to its high viscosity, handling may be difficult for certain applications. It is expensive, which may be prohibitive to use in certain low-cost applications. Variability in price and quality of this biologically-produced substance means it is also an unpredictable raw material, which is undesirable to manufacturers looking to make consistent products. The amount of DHA and MGO present may vary depending on the source of the honey, meaning its antimicrobial properties can be unpredictable. There exists a need for a product that may fulfil the function of Manuka honey without these drawbacks.

Glycerol is a common component of many formulations, including food, pharmaceuticals and cosmetics. The present invention provides a composition, referred to here as “active glycerol”, comprising glycerol and the active compounds found in manuka honey, including DHA and MGO. As a result, the composition has antimicrobial properties. By using glycerol as the bulk medium, the composition is acceptable for use in many products. Given glycerol is abundant and low cost, compositions of the invention can be provided at a low-cost with consistent properties and a predictable supply chain.

Also provided herein is a method of making the composition that uses a simple chemical process.

### SUMMARY

According to the present invention there is provided an antimicrobial composition comprising glycerol and dihydroxyacetone.

Preferably, the antimicrobial composition further comprises one or more compounds selected from: methylglyoxal, glycolic acid, formic acid, acetic acid, 2-propanol, acetone, glyceraldehyde and acrolein; preferably methylglyoxal.

Conveniently, the dihydroxyacetone content is between about 150 mg kg⁻¹ and 4000 mg kg⁻¹.

Advantageously, the methylglyoxal content is between about 0 mg kg⁻¹ and 1200 mg kg⁻¹.

Preferably, the pH of the composition is lower than about 4.0.

Conveniently, the composition has at least one of the following properties: antibacterial, antifungal, antiviral and biocidal.

Advantageously, the composition has a non-peroxide activity against Staphylococcus aureus of more than 15% w/v phenol equivalent.

Preferably, the composition is antibacterial, characterised by a minimum inhibitory concentration of 10% or less against bacteria.

Conveniently, the composition is antifungal, characterised by a minimum inhibitory concentration of 10% or less against fungi.

Advantageously, the antimicrobial composition is food grade.

According to the present invention, there is provided a method of making the antimicrobial composition of the invention, comprising mixing: a reagent comprising glycerol and an oxidant; optionally with a catalyst.

Preferably, the oxidant comprises hydrogen peroxide.

Conveniently, the catalyst comprises ferrous (II) lactate.

Advantageously, the antimicrobial composition is for use to prevent the growth of, or kill, at least one of: bacteria, yeasts, fungi and viruses.

According to the present invention, there is provided the use of the antimicrobial composition of the invention in the treatment of a wound, wherein a product comprising the composition is applied to the wound.

According to the present invention, there is provided a wound dressing comprising the antimicrobial composition of the invention.

According to the present invention, there is provided a food product comprising the antimicrobial composition of the invention.

According to the present invention, there is provided a cosmetic product comprising the composition of the invention.

According to the present invention, there is provided a cleaning product comprising the composition of the invention.

According to the present invention, there is provided the use of the antimicrobial composition of the invention in the prevention or treatment of a skin condition.

According to the present invention, there is provided the use of the antimicrobial composition of the invention in therapy.

### DETAILED DESCRIPTION

### Compositions of the Invention

Disclosed herein is an antimicrobial composition comprising glycerol (also known as glycerine), dihydroxyacetone (DHA) and optionally one or more compounds selected from: methylglyoxal (MGO), glycolic acid, formic acid, acetic acid, 2-propanol, acetone, glyceraldehyde and acrolein. “Antimicrobial” refers to the effect of reducing or preventing the growth of microorganisms or killing microorganisms. Antimicrobial may include one or more of the following: antibacterial, antifungal, antiviral and biocidal/bactericidal.

In some embodiments, the composition comprises between about 200 to about 4000 mg of DHA per kg, or between about 1000 to about 3500 mg of DHA per kg, or between about 1500 to about 3000 mg of DHA per kg. Preferably, the composition comprises between about 1500 to about 2500 mg of DHA per kg. More preferably, the composition comprises about 2200 mg of DHA per kg.

In some embodiments, the composition comprises between about 50 to about 1200 mg of MGO per kg, or between about 100 to about 750 mg of MGO per kg or between about 150 to about 400 mg of MGO per kg. Preferably, the composition comprises between about 150 to about 250 mg of MGO per kg. More preferably, the composition comprises about 220 mg of MGO per kg.

The composition may be made via the chemical processes disclosed herein. As a result, in some embodiments, the composition may further comprise a catalyst. In some embodiments, the composition is substantially free of catalyst. In some embodiments, the composition comprises less than 100 mg kg⁻¹ catalyst, or less than 50 mg kg⁻¹. In some embodiments, the composition comprises between about 20 to 40 mg kg⁻¹ catalyst.

In some examples, the composition further comprises additional compounds. In some examples, the composition comprises one or more of: glycolic acid, oxalic acid, acetone, propan-2-ol, glyceraldehyde, acetic acid, formic acid, lactic acid, 3-hydroxypropionaldehyde (3-HPA) and acrolein. Advantageously, glycolic acid, oxalic acid, propan-2-ol, acetic acid, formic acid, lactic acid, 3-hydroxypropionaldehyde (3-HPA) and acrolein are known to have antimicrobial effects. These compounds may increase the antimicrobial properties of the composition. In some examples, the presence of these compounds means that a lower level of MGO is required to achieve a given level of antimicrobial activity. In some examples, the presence of these compounds means that no MGO is required to achieve a given level of antimicrobial activity. Compositions comprising additional antimicrobial compounds may also be more effective in reducing or preventing the growth of a wider range of microorganisms. The presence of more than one compound in the glycerol compositions can provide synergistic antimicrobial activity.

In some embodiments, the composition comprises about 1000 to 5000 mg of glycolic acid per kg, preferably about 2000 to 3000 mg of glycolic acid per kg, more preferably about 2500 mg of glycolic acid per kg.

In some embodiments, the composition comprises about 1000 to 5000 mg of formic acid per kg, preferably about 2000 to 3500 mg of formic acid per kg, more preferably about 2800 mg of formic acid per kg.

In some embodiments, the composition comprises about 400 to 2000 mg of acetic acid per kg, preferably about 600 to 1000 mg of acetic acid per kg, more preferably about 900 mg of acetic acid per kg.

In some embodiments, the composition comprises about 1500 to 7000 mg of propan-2-ol per kg, preferably about 2500 to 4000 mg of propan-2-ol per kg, more preferably about 3200 mg of propan-2-ol per kg.

10 In some embodiments, the composition comprises about 1000 to 4000 mg of acetone per kg, preferably about 1500 to 3000 mg of glycolic acid per kg, more preferably about 2000 mg of acetone per kg.

In some embodiments, the composition comprises about 2200 mg per kg of DHA and about 250 mg per kg of MGO. In some embodiments, the composition comprises about 2200 mg per kg of DHA, about 250 mg per kg of MGO and at least one further component. For example, the composition further comprises (per kg of the composition): about 2527 mg glycolic acid and/or about 2850 mg formic acid and/or about 885 mg acetic acid and/or about 3265 mg 2-propanol and/or about 1902 mg acetone. In a preferred embodiment, the composition comprises (per kg of the composition): 2200 mg DHA, about 250 mg MGO, about 2527 mg glycolic acid, about 2850 mg formic acid, about 885 mg acetic acid, about 3265 mg 2-propanol and about 1902 mg acetone. In another preferred embodiment, the composition comprises (per kg of the composition): 1125 mg DHA, about <50 mg MGO, about 2527 mg glycolic acid, about 2850 mg formic acid and about 885 mg acetic acid.

Glycerol of any grade may be used. In some examples, the glycerol may be USP grade or food-grade glycerol. In some examples, the glycerol fulfils the requirements of EC Directive 2007/68/EC and contains no allergenic substances as listed in Appendix III of the Directive.

Glycerol is a viscous liquid that is miscible with water. Aqueous solutions of glycerol are known to vary in viscosity as a function of their water content. In some examples, the composition further comprises water. The amount of water may be sufficient to reduce the viscosity of the composition relative to that of pure glycerol. To make the composition suitable for use in certain applications, a composition of a particular viscosity may be preferred, for example, to make the composition easier to handle. The amount of water may be increased or decreased as required to achieve a desired level for a particular use or application. In some embodiments, the water content of the composition may be less than 10 wt %, or less than 8 wt % or less than 5 wt % or less than 3 wt % or less than 2 wt %. In some embodiments, the water content of the composition may be between about 5 wt % and about 15 wt %.

The pH of pure glycerol is slightly acidic and may be around 6-7. Compositions of the present invention may have a pH of about 5 or less. In some examples, they have a pH of less than about 4. Preferably, the composition has a pH between about 2.5 and about 3.5. More preferably, the composition has a pH of about 3.0.

### Antimicrobial Properties

Antimicrobial substances reduce or eliminate growth of microbes. Antibacterial substances reduce or eliminate growth of bacteria. Antifungal substances reduce or eliminate growth of fungi. Antiviral substances reduce or eliminate growth of viruses. Biocidal substances cause microbial death. The terms biocidal and bactericidal may be used interchangeably. In some embodiments, the composition may be antibacterial and/or antifungal and/or antiviral and/or biocidal. Preferably, the composition has one or more properties selected from: antibacterial, antifungal, antiviral and biocidal. More preferably, the composition is antibacterial, antifungal, antiviral and biocidal.

The non-peroxide activity (NPA) of a sample can be measured to quantify its antimicrobial properties. This is a common analytical method used to measure the antimicrobial properties of Manuka honey. Hydrogen peroxide has antimicrobial properties and in some circumstances is produced naturally by enzymes in honey. An enzyme catalase is added to remove hydrogen peroxide from the sample before measuring the NPA. Manuka honey typically has a high NPA relative to other honeys, which is attributed to the presence of a high concentration of MGO.

Compositions of the present invention may have a NPA against a bacterium, such as Staphylococcus aureus, of more than 15% w/v phenol equivalent. The NPA against a bacterium, such as Staphylococcus aureus, may be between 15 and 25% w/v phenol equivalent, or preferably between 18.5 and 21.5% w/v phenol equivalent. More preferably, the composition has a non-peroxide activity against a bacterium, such as Staphylococcus aureus, of about 20% w/v phenol equivalent. In some examples the composition has an NPA of more than about 12% w/v phenol equivalent and is considered medical grade or other grade. Preferably, the composition has an NPA of about 20% w/v phenol equivalent or more.

In some examples, the composition is capable of limiting growth of one or more types of microorganism. For example, the composition is capable of limiting growth of a Gram-positive bacterium, such as Staphylococcus aureus and/or a Gram-negative bacterium, such as Pseudomonas aeruginosa and/or a fungus, such as a yeast Candida albicans or a mould/filamentous fungus such as Aspergillus niger. The minimum inhibitory concentration (MIC) quantifies the lowest level of an antimicrobial substance that greatly inhibits growth of a microorganism. This may be measured using the industry standard test CLSI M07. In some examples, the composition of the present invention has a MIC of 10% or less, or 7.5% or less, or 5% or less, or 2.5% or less, measured against at least one of: a Gram-positive bacteria, a Gram-negative bacteria, a yeast and a mould/filamentous fungus. Preferably, the composition of the present invention has a MIC of 10% or less, or 7.5% or less, or 5% or less, or 2.5% or less, measured against all of: a Gram-positive bacteria, a Gram-negative bacteria, a yeast and a mould/filamentous fungus.

Compositions of the present invention may be capable of causing microbial death of one or more types of microorganism. The minimum bactericidal concentration (MBC) quantifies the lowest level of antimicrobial agent that results in microbial death. For example, the MBC is measured against a Gram-positive bacterium, such as Staphylococcus aureus and/or a Gram-negative bacterium, such as Pseudomonas aeruginosa and/or a fungus, such as Candida albicans (yeast) and/or Aspergillus niger (mould/filamentous fungus). This may be measured using the industry standard test CLSI M26-A. In some examples, the composition of the present invention has a MBC of 10% or less, or 7.5% or less, or 5% or less, or 2.5% or less, measured against at least one of: a Gram-positive bacteria, a Gram-negative bacteria, a yeast and a mould/filamentous fungus. In some examples, the composition of the present invention has a MBC of 10% or less, or 7.5% or less, or 5% or less, or 2.5% or less, measured against all of: a Gram-positive bacteria, a Gram-negative bacteria, a yeast and a mould/filamentous fungus.

The kill time of the composition may be measured using industry standard ASTM E2783. For example, that may be measured against Gram-positive bacterium, such as Staphylococcus aureus, a Gram-negative bacterium, such as Pseudomonas aeruginosa, a fungus, such as a yeast Candida albicans or a mould/filamentous fungus such as Aspergillus niger. In some examples, the composition has a 3-log kill time of less than 10 minutes, or between 5 and 10 minutes.

The compositions of the invention have preservative properties and can be used in a range of products and applications.

### Method of Making the Composition

The composition may be made by admixing the components of the composition. Advantageously, the inventors have found that compositions of the present invention can also be made via a simple method involving a chemical process. This chemical process comprises at least one chemical reaction of glycerol to at least partially convert glycerol into at least one product. Some of the compounds that can be made via this chemical process are antimicrobial, so the process can be used to make compositions of the present invention.

The method comprises a step or a series of steps. These steps may include: making a reagent mixture comprising glycerol, an oxidant and optionally a catalyst; maintaining the mixture at a particular temperature for a period of time. The method may comprise further additional steps. In some embodiments the reagent mixture comprises a catalyst. In some embodiments, the reagent mixtures does not comprises a catalyst. As will be explained below, both of these methods produce compositions with antimicrobial activity. The compositions produced by each method will generally have similar but differing components. The use of a catalyst in the reagent mixture will favour the production of methylglyoxal (MGO). The beneficial antimicrobial activity is thought to be a result of the compounds produced by the method.

The method involves a chemical process comprising at least one chemical reaction. It has been found by the inventors that reactions of glycerol start upon mixing the reagents, even at room temperature. In some examples, the process comprises at least one oxidation reaction to form DHA via oxidation of glycerol. In some examples, the process comprises at least one dehydration reaction. MGO can be formed by dehydration of DHA. In some examples, the chemical process comprises at least one photochemical reaction. In some examples, the chemical process comprises at least one photochemical oxidation reaction. In some examples, the chemical process is carried out under UV light.

In some embodiments, the compositions made by the chemical process comprise glycerol and DHA. They may further comprise MGO. In some embodiments, they comprise at least one additional product selected from: glycolic acid, oxalic acid, acetone, propan-2-ol, glyceraldehyde, acetic acid, formic acid, lactic acid, 3-hydroxypropionaldehyde (3-HPA) and acrolein.

The reagents may be added in any order. Stirring is not required, however, the reagents may be stirred or otherwise agitated after or during addition. For example, the oxidant and the catalyst may be added to glycerol and the glycerol may be stirred during addition. Preferably, the reagents are stirred until they are thoroughly mixed. Stirring may be maintained throughout the method, but it is not required.

During the chemical process, the reagent mixture is maintained at a temperature for a period of time. In some examples, the temperature is room temperature (for example at about 20° C.). In some examples, the reagent mixture may be heated and the temperature maintained above room temperature. In some examples, the temperature may be slightly above room temperature (for example, about 25° C.). In some examples, the temperature is maintained at higher temperatures. For example, the temperature is maintained at 30° C., 35° C., 40° C. or 50° C. Preferably, the reaction is heated to about 37° C. As the reactions start upon mixing the reagents and the concentration of products builds over time, a range of time periods are suitable. In some embodiments, the time may be less than 1 hour or about 1 hour or about 5 hours or about 24 hours or about 48 hours or about 72 hours. In some examples, the time may be longer than 72 hours.

The exposure of the mixture to light during the chemical process may be controlled. For example, it may be carried out under ambient light, in the dark or under UV-light irradiation. Preferably, the reaction is carried out in an incubator at 37° C. in the dark for 24 hours. More preferably, the reaction is carried out in an incubator at 37° C. in the dark for 48 hours. The reaction may be conducted in a sealed or open vessel. The pressure in the reaction vessel may be atmospheric pressure or it may be above atmospheric pressure. Regular periodic venting of the vessel at least every 24 hours may benefit the antimicrobial potency of the composition.

The reagents comprise an oxidant. For example, common oxidants such as gaseous molecular oxygen or hydrogen peroxide may be used. In some embodiments, an aqueous solution comprising an oxidant may be used. For example, the oxidant may comprise hydrogen peroxide or an aqueous solution of hydrogen peroxide. The concentration of the hydrogen peroxide solution may be 2 to 98% or between 2 to 50% or between 20 to 35%. In some embodiments, the hydrogen peroxide solution concentration may be about 30%. The oxidant is preferably a food grade material, such as aqueous hydrogen peroxide solution.

In some examples, the amount of hydrogen peroxide used is less than 100 g per kg of glycerol. In some examples, the amount of hydrogen peroxide used is less than 50 g per kg of glycerol. In some examples, the amount of hydrogen peroxide used is between 10 to 50 g per kg of glycerol or between 20 to 35 g per kg. When an aqueous solution of hydrogen peroxide is used, the volume of solution added is adjusted based on the concentration to achieve the correct ratios of hydrogen peroxide to glycerol. For example, 100 g of a 30% hydrogen peroxide solution contains 30 g of hydrogen peroxide. Preferably, a 30% aqueous solution of hydrogen peroxide is used in a ratio between about 50 to 150 ml and more preferably, about 80 ml per kg of glycerol.

Suitable catalysts for use in the method comprise metals and/or metal compounds. For example, the catalyst may comprise a metal salt. Suitable metals include iron, copper, gold and silver. Preferably, the catalyst comprises iron or copper, and more preferably iron. For example, the catalyst comprises an iron salt. The catalyst may be in any form. For example, a powder, or a solution such as an aqueous solution. Preferably, the catalyst comprises ferrous (II) lactate. For example, an aqueous solution comprising ferrous (II) lactate may be used. In some examples, the catalyst may be food grade.

In some examples, the amount of catalyst used is less than 1 g per kg of glycerol or less than 100 mg per kg of glycerol or less than 50 mg per kg of glycerol. In some examples, the amount of catalyst used is between 20-40 mg per kg of glycerol. In some examples, the amount of catalyst used is between 30-35 mg per kg of glycerol. In some embodiments, an aqueous solution of catalyst is used, and the volume of the solution is adjusted to achieve these ratios of active catalyst to glycerol. In some embodiments, a 1% aqueous solution of Ferrous (II) lactate is used and the volume of the solution can be adjusted to achieve the above ratios of Fe(II) to glycerol. The ratio may be less than 100 ml of 1% Ferrous (II) lactate solution per kg of glycerol, or less than 75 ml, or less than 50 ml. Preferably, the ratio is in the range of about 5 to 30 ml per kg of glycerol. Most preferably, 16 ml of a 1% aqueous solution of Ferrous (II) lactate is used per kg glycerol, resulting in about 31 mg of Fe(II) per kg glycerol.

The method may comprise a further step to cool the mixture. The temperature of the mixture may be reduced to room temperature or lower, for example, 15° C., or 10° C., or 5° C., 0° C., or −5° C. or −10° C. In some examples, the mixture may be transferred to a freezer.

In some examples, the method of making the composition comprises additional steps to alter the composition obtained from the chemical process. Examples of said additional steps include reducing the water content of the composition, adding additional components to the composition and removing at least some of the catalyst, if present. The addition of further components may be used to add compounds that were not made from the chemical process or to increase the concentration of compounds that were made. Suitable compounds to be added include DHA, MGO or any of the earlier disclosed active compounds.

As mentioned above, the method of the invention can optionally use a catalyst. Compositions which result from a method using a catalyst contain MGO which contributes to antimicrobial activity. In comparison, compositions which result from a method which does not use a catalyst contain lower levels of MGO, or a negligible level of MGO. Both types of compositions contain DHA which contributes to antimicrobial activity. Both types of compositions may contain organic acids (such as formic, glycolic and acetic acids) which contribute to antimicrobial activity.

As described, the composition may comprise water and it may be advantageous for the composition to have a particular water content. In some embodiments, the method of making the composition further comprises reducing its water content. This can be done by any conventional means, for example, drying under a vacuum such as rotary evaporation or distillation, drying in a desiccator or by the addition of a drying agent.

In some examples, the composition may be stored at or below room temperature. In some examples, it is advantageous to store the composition at lower temperatures, for example, 15° C., or 10° C., or 5° C., 0° C., or −5° C. or −10° C. In some examples, the composition may be stored in a freezer. Storing compositions of the invention at low temperature, for example below 0° C., allows for an increase in potency. This may be due to an increase of compounds in the composition which result in antimicrobial activity. For example, storage of compositions of the invention below 0° C. may increase the level of DHA, which in turn converts to MGO.

### Uses of the Composition

The compositions of the invention are suitable to be used as glycerol replacements. It may be used to partially, or fully, replace glycerol in existing formulations. The compositions of the present invention are suitable for use in a range of different formulations, such as in food, cosmetics or pharmaceuticals. Advantages of the use of the composition in existing formulations and new products include direct replacement of glycerol, low cost, ease of handling, water miscibility, antimicrobial properties, preservative properties and improved shelf life.

Advantageously, the composition provides improved preservative properties compared with glycerol. This may lead to a reduction in the amount required in a formulation without loss of shelf life. It may lead to improved shelf life if glycerol is directly replaced with the same amount of the composition of the invention. The antimicrobial properties of the composition can make existing formulations antimicrobial without the need for significant reformulation. The composition may be perceived as containing natural ingredients and can be produced vegan. It may replace existing preservatives, such as parabens, that may be undesirable to consumers. The composition is easy to handle, water miscible, so is easy to incorporate into water-, oil- or water and oil-based formulations (with the use of other components such as emulsifiers as needed).

The composition of the present invention is suitable for use in cosmetics. For example, in make-up, lipsticks, moisturisers, biogels, face creams, body lotions and skin firmers. The composition of the invention is also suitable for use in toiletries such as hand wash, face wash, soaps, shower gels and shampoos. In some examples, the composition is suitable for use in the treatment of skin conditions such as acne by applying the composition or a formulation comprising the composition to the skin. Advantageously, the antimicrobial properties of the composition may reduce the levels of microbes present on the skin, which may reduce the occurrence of acne. Glycerol is commonly used as a preservative. However, this requires high amounts of glycerol (e.g. up to 60 to 70%) for optimum preservative properties. At these concentrations, formulations can become very hygroscopic, which can be undesirable in products such as moisturisers. As a result, glycerol is usually not used at levels that achieve optimum preservative properties. The antimicrobial properties of the composition of the invention may achieve the desired preservation and shelf life at lower concentrations that do not lead to these undesirable properties.

In some examples, the composition is food grade and is suitable for use in food formulations and food products. This may also make it suitable for use in ingestible pharmaceutical formulations. There is a growing trend for producing “active”, “medicinal” or functional foods. The composition may be used in food products to provide antimicrobial properties or as a preservative to improve shelf life.

Glycerol is used as an ingredient in a variety of food and beverage products to help retain moisture, prevent sugar crystallization, and add bulk, smoothness, softness, sweetness and texture. Like most sugar alcohols, glycerol is not as sweet as sugar-it is about 60-75% as sweet. Glycerol is also widely used in the food industry, for example to refine flavours, improve the consistency, or optimize preservation. Glycerol E-number, E 422, acts as a solvent for food colourings and flavourings in several soft drinks or confectionary. Glycerol can also be plant based and as a result suitable for vegans. The FDA classifies glycerol as generally recognized as safe (GRAS) when used as a food additive.

The sweet taste of glycerol is one of its most recognisable characteristics. Glycerol is 60% as sweet as other refined sugars, with lower calorie percentage per teaspoon. Glycerol is used widely as an artificial sweetener, it is added to most processed foods in varying quantities, including ice cream and chewing gum. It is used widely in baking for fondant and icing.

The composition of the present invention is suitable for use in the treatment of wounds. Advantageously, the composition may promote wound healing by reducing or preventing the growth of a range of microbes at the wound site, such as bacteria, fungi and viruses. Advantageously, this may reduce the occurrence of infection at the wound site. For example, the composition, or a product comprising the composition, may be used to cleanse wounds or used in the treatment of infected wounds. The composition of the present invention may be used to at least partially or completely replace glycerol in existing formulations used in the treatment of wounds. The antimicrobial properties of the composition of the invention may promote wound healing.

Traditionally, antimicrobials are used in wound dressings. These dressings may be suitable for the treatment of wounds or burns. Common examples of antimicrobials used in these dressings are silver and polyhexamethylene biguanide (PHMB). Silver has an antimicrobial effect but has been shown to inhibit healing if it accumulates excessively at the wound site. PHMB is a known category 2 carcinogen. Compositions of the present invention are suitable for use as a replacement for antimicrobials such as silver or PHMB in wound dressings.

Wound dressings comprising antimicrobials suitable for use in conjunction with the present invention can have various forms. In some examples, the dressing comprises multiple layers, one of which comprises one or more antimicrobials. For example, the external layer that is in contact with the wound comprises an antimicrobial and the dressing consists of additional layers that provide structure to the dressing. In some examples, the antimicrobial is applied as a coating on the surface of the dressing in contact with the wound. In some examples, wound dressings consist of essentially one layer comprising an antimicrobial. For example, the dressing comprises a sheet or gauze which is impregnated with an antimicrobial.

Other common wound-healing products include gel-based formulations, such as hydrogel-based formulations. These can come in various forms including amorphous gels, amorphous gel sheets, gel tubes, and conventional sheet or gauze dressings impregnated with gels. Commonly, these gel formulations comprise glycerol. Direct replacement of glycerol with the composition of the invention in hydrogel formulations advantageously provides additional antimicrobial properties whilst retaining the physical properties of the gel.

Also disclosed herein is a method of promoting wound healing comprising applying the composition to a wound. In some embodiments, a formulation comprising the composition is applied to a wound. In some embodiments, a dressing comprising the composition is applied to a wound. In some embodiments, a hydrogel formulation comprising the composition is applied to a wound. In some embodiments, a dressing impregnated with a hydrogel formulation comprising the composition is applied to a wound. In some examples, the method comprises multiple steps, such as applying the composition of the present invention in conjunction with a conventional wound dressing. For example, the composition or a formulation comprising the composition is applied to a wound, followed by applying a dressing to cover the wound. In some examples, the dressing is a conventional sheet dressing. In some examples, the dressing also comprises the composition of the present invention.

The compositions of the invention can also be used in the prevention or treatment of skin conditions including eczema, dermatitis, dry skin, cracked heels and rosacea. The compositions of the invention can also be used in the treatment of cerebral edema (previously treated using glycerol).

The compositions of the invention can also be used in cleaning products such as surface sprays and wipes. These products can be used in the home and in other situations such as clinical or industrial settings. In some uses, a product comprising a composition of the invention may be applied to a surface (for example by wiping or spraying) and left for a period of time (such as about 5 minutes) before removed or before the surface is deemed clean.

In general, the composition of the invention compositions of the invention can be used to replace glycerol where used in conventional products and processes. This can include use in products such as moisturizer, shampoo, dishwasher detergent, toilet cleaning gel, toothpaste, laundry detergent, shaving cream, cleaning wipes, septic tank additives, printer ink cartridges, air freshener, candles, hair colours, makeup and antifreeze.

One particular use in the prevention and/or treatment of throat infections. Some traditional products and treatments contain glycerol and/or honey. The compositions of the invention can be used in such products and treatments, such as in throat lozenges and cough syrups. The properties of the compositions of the invention make them well suited for these uses.

The sweet taste of glycerol is one of its most recognisable characteristics. Glycerol is 60% as sweet as other refined sugars, with lower calorie percentage per teaspoon. Glycerol is used widely as an artificial sweetener, it is added to most processed foods in varying quantities, including ice cream and chewing gum. It is used widely in baking for fondant and icing.

Glycerol's capacity as an emulsifier is strengthened by its humectant properties, which are what lends bread, pastries and other baked products their moistness. It also helps to preserve foods.

The compositions of the invention can be used to replace glycerol to provide any or all of these desired properties while also providing additional beneficial properties, such as antimicrobial activity and/or preservative effects.

The compositions of the invention may be used to replace the traditional use of glycerol in food products or processes, such as in condensed milk, whey products, puddings, clotted cream, processed fruits and vegetables (dried or canned vegetables or fruits, precooked vegetables), precooked pasta, rolled oats, breakfast cereals, rice or tapioca pudding, breading or batters, precooked rice products, and sweets including marshmallows. Other products where the compositions of the invention may be used to replace the traditional use of glycerol include gelatine products.

The composition of the invention can also be used in clinical and surgical settings, both as a replacement for the traditional use of glycerol, and also in new products and processes. One example of this relates to cerebral edema, which happens when there is an excess accumulation of fluid in the intra-cellular spaces if the brain. It is usually caused by a brain trauma, cancer or stroke. Because of the hygroscopicity, glycerol is used in IV fluids in order to reduce excessive intracranial pressure caused by cerebral edema. It does this by drawing out excess fluid from the body tissue and bloodstream, which it is able to do by being extremely hygroscopic. It also dehydrates the tissue by preventing the kidneys from reabsorbing water. This reduces the volume of blood which ultimately reduces intracranial pressure. Compositions of the invention can be used in the treatment of conditions such as cerebral edema with the added benefits of antimicrobial activity and/or preservative effects.

### Example 1—25 g Scale

All reagents used are commercially available. For example, hydrogen peroxide can be purchased from ThermoFisher Scientific and Ferrous (II) Lactate can be purchased from Sigma Aldrich.
1) In a 50 ml polypot add 0.4 ml 1% aqueous Ferrous (II) Lactate to 25 g glycerol.
2) Add 2 ml H₂O₂ (30% aqueous solution).
3) Mix thoroughly.
4) Store the reaction solution in the dark at 37° C.

Samples were taken from the mixture at various times and the levels of DHA and MGO were quantified by HPLC-UV. Comparative data including that of blank glycerol is shown in Table 1. The sample taken at t=0 days was taken immediately after thorough mixing of the reagents (between steps (3) and (4) of the above method).

TABLE 1
DHA and MGO levels over the course of a 35-
day reaction time for a 25 g scale reaction.

| t / days | DHA / mg kg⁻¹ | MGO / mg kg⁻¹ |
|---|---|---|
| Glycerol standard | <150 | <50 |
| 0 | 1174 | <50 |
| 1 | 2752 | 372 |
| 2 | 2787 | 320 |
| 3 | 2654 | 328 |
| 4 | 2542 | 326 |
| 5 | 2354 | 298 |
| 10 | 2196 | 254 |
| 14 | 2043 | 251 |
| 35 | 1578 | 228 |

### Example 2—1 kg Scale

1) In a 1 kg opaque food-grade plastic tub, add 16 ml 1% aqueous Ferrous (II) Lactate to 1 kg glycerol.
2) Add 80 ml H₂O₂ (30% aqueous solution) whilst mixing using a plastic or glass stirrer.
3) Mix thoroughly and seal the container.
4) Place the sample in a 37° C. incubator in the dark.

Samples were taken from the mixture at various times and the levels of DHA and MGO were quantified by HPLC-UV. Comparative data including that of blank glycerol is shown in Table 1. The sample taken at t=0 days was taken immediately after thorough mixing of the reagents (between steps (3) and (4) of the above method).

TABLE 2
DHA and MGO levels over the course of a 4-day
reaction time for a 1 kg scale reaction.

| t / days | DHA / mg kg⁻¹ | MGO / mg kg⁻¹ |
|---|---|---|
| Glycerol standard | <150 | <50 |
| 0 | 206 | <50 |
| 1 | 2230 | 207 |
| 2 | 2078 | 355 |
| 3 | 1969 | 332 |
| 4 | 1988 | 300 |

### Example 3—Non-Peroxide Activity Plate Assay Testing

In this test, the enzyme catalase was added to the sample to rapidly break down hydrogen peroxide to water and oxygen. In the standard NPA plate assay, an excess of this enzyme is added during sample preparation to eliminate all antimicrobial activity in a sample due to the presence of hydrogen peroxide, hence only non-peroxide activity in a sample is measured. The test was carried out using a Gram-positive bacteria, Staphylococcus aureus. The result is a value in % w/v relative to phenol standards. Two honey samples and several control samples consisting of glycerol with additional components were tested alongside the composition of the invention samples and the results are shown in Table 4. Samples were run in duplicate or triplicate and the mean result calculated.

4 samples of the composition of the invention were tested:
Sample 1: a freshly made sample
Sample 2: stored for 234 days in a 37° C. incubator
Sample 3: stored for 234 days at room temperature
Sample 4: stored for 234 days in a fridge
Sample 5: stored for 234 days in a freezer

TABLE 3
NPA assay results for the composition of
the invention compared with Manuka honey.

| | | | Mean Result / |
|---|---|---|---|
| | DHA / mg | MGO / mg | % w/v phenol |
| Sample | kg⁻¹ | kg⁻¹ | equivalent |
| Honey (control) | Not known | Not known | 13.0 |
| Manuka Honey | Not known | Not known | 22.0 |
| (Medical Grade) | | | |
| Glycerol | Not known | Not known | <9.5 |
| Invention sample 1 | 3111 | 167 | 18.5 |
| Invention sample 2 | 236 | 191 | 20.0 |
| Invention sample 3 | 1549 | 256 | 17.0 |
| Invention sample 4 | 1989 | 251 | 18.0 |
| Invention sample 5 | 2427 | 207 | 19.5 |
| Glycerol + H₂O₂ + water | N/A (none) | N/A (none) | <9.5 |
| Glycerol + catalyst + water | N/A (none) | N/A (none) | <9.5 |

### Example 4—Minimum Inhibitory Concentration (MIC) and Minimum Bactericidal Concentration (MBC)

MIC and MBC testing was carried out to industry standards CLSI M07 and CLSI M26-A, respectively.

Sample: Fresh composition was made according to the invention, where Composition A was tested soon after manufacture and Composition B was tested approximately one year after manufacture.

MIC and MBC of this sample were measured against the following test organisms: Staphylococcus aureus (S. aureus) (ATCC 6538), Pseudomonas aeruginosa (P. aeruginosa) (ATCC 15442), Candida albicans (C. albicans) (ATCC 10231) and Aspergillus brasiliensis (A. brasiliensis) (ATCC 16404).

The test data show that compositions of the invention (as exemplified by Composition A) are effective at inhibiting growth of bacteria (S. aureus; P. aeruginosa) against the two different representative types of bacteria (Gram +ve and Gram −ve) at 2.5% V/V, yeast (C. albicans) at 10.0% V/V and filamentous fungi—mould—(A. brasiliensis) at 5.00% V/V.

This data also shows that compositions of the invention are biocidal (irreversibly killing) at similar concentrations to its growth inhibitory activity against bacteria and yeast. This is an additional and important antimicrobial activity not exhibited by glycerol itself.

The results from Composition B shows that compositions of the invention remain effective over a period of at least approximately one year (as tested, but this period will be longer) from manufacture, although some activity is lost.

TABLE 4
MIC and MBC assay results for S. aureus, P. aeruginosa,
C. albicans and A. brasiliensis in the composition
of the invention (Composition A and Composition B).

| | | |
|---|---|---|
| Organism | MIC / % | MBC / % |

Composition A

| | | |
|---|---|---|
| Staphylococcus aureus | 2.50% | 5.00% |
| Pseudomonas aeruginosa | 2.50% | 2.50% |
| Candida albicans | 10.00% | 10.00% |
| Aspergillus brasiliensis | 5.00% | >5.00% |

Composition B

| | | |
|---|---|---|
| Staphylococcus aureus | 5.00% | 10.00% |
| Pseudomonas aeruginosa | 10.00% | 10.00% |
| Candida albicans | 10.00% | 10.00% |
| Aspergillus brasiliensis | 10.00% | >10.00% |

### Example 5—Kill-Time Assays

Kill-time assays were carried out to industry standards, ASTM E2783-22. This assay measures how quickly compositions of the invention kill strains at 100.0% V/V concentration. Two standard bacterial strains are included (Escherichia coli (Gram-ve) and Staphylococcus aureus (Gram+ve)), two drug resistant strains that have multiple resistance to antibiotics (Klebsiella pneumoniae CRE and Enterococcus faecalis VRE) to show effectiveness against these strains- and the yeast Candida albicans. Candida albicans is a different class of micro-organism (eukaryotic) compared to bacteria.

### Kill-Time in the Range of 10 Minutes to 24 Hours

In this example, kill-time assays compare the biocidal (irreversible killing) efficacy of compositions of the invention at 100.0% V/V with time in a range of 10 minutes to 24 hours, compared to sterile water.

TABLE 5
Kill time assay results for Staphylococcus aureus,
Escherichia coli, Klebsiella pneumoniae (CRE), Enterococcus faecalis
(VRE) and Candida albicans.

| | | | | | | Log Reduction, |
|---|---|---|---|---|---|---|
| | | Log Reduction, R, in | | | | R, in Sterile |
| | | Composition of Invention | | | | Water |
| | Strain | 10 | 1 | 4 | 24 | 24 |
| Organism | ID | mins | hour | hours | hours | hours |
| Staphylococcus | ATCC | >3.42 | >3.42 | >3.42 | >3.28 | 0.14 |
| aureus | 6538 | | | | | |
| Escherichia coli | ATCC | >3.39 | >3.39 | >3.39 | >3.47 | 0.26 |
| | 10536 | | | | | |
| Klebsiella | DSM | >3.33 | >3.33 | >3.33 | >3.26 | −0.1 |
| pneumoniae | 2637 | | | | | |
| (CRE) | | | | | | |
| Enterococcus | DSM | >2.70 | >3.50 | >3.50 | >3.40 | −0.11 |
| faecalis (VRE) | 51575 | | | | | |
| Candida | ATCC | >3.39 | >2.87 | >3.39 | >3.08 | 1.36 |
| albicans | 10231 | | | | | |

According to ASTM E2783-22, the composition of the invention produced the above log reductions (Table 5) across three replicates when tested at 100.0% of the working concentration after 10 minutes, 1 hour, 4 hours and 24 hours at 25° C. against a set of standard and medically important drug-resistant strains, including CRE and VRE.

The kill rate is compared to sterile distilled water. These data show that compositions of the invention achieve minimum>3 log reduction against bacteria and yeast in at least 10 minutes and against the bacterium Enterococcus faecalis after at least 60 minutes. The kill-time values are as measured as tested.

The tested compositions of the invention kill rapidly (at 100.0% V/V), in general within 10 minutes by at least 3 logs (as measured, but this will be higher in practice). This puts compositions of the invention in the category of an effective antimicrobial agent.

Compositions of the invention kill drug resistant strains of bacteria, including CRE and VRE. This therefore shows that these compositions have additive capability against these medically important strains in such formulations as antiseptics for topical application, toothpastes and in wound dressings.

The additional activity against drug-resistant strains shows that the compositions can additionally combat the development of antimicrobial resistance (AMR).

### Kill-Time in the Range of 30 Seconds to 5 Minutes

This assay was also used to compare the biocidal (irreversible killing) efficacy of compositions of the invention at 100.0% V/V with time in a range of 30 seconds to 5 minutes, compared to the initial inoculum (Table 6).

Two standard bacterial strains are included (Escherichia coli (Gram −ve) and Staphylococcus aureus (Gram +ve), with Methicillin resistance),

The kill rate is compared to the initial inoculum. This data show that compositions of the invention achieve approximately a 4-log reduction against both bacterial strains within 5 minutes.

TABLE 6
Kill time assay results for MRSA Staphylococcus aureus
and Escherichia coli.

| Initial inoculum | | | Log |
|---|---|---|---|
| (CFU per mL) | Time | CFU per mL | Reduction (R) |
| MRSA | | | |
| 7.50 × 10⁷ (Time 0) | | | |
| Sample 1 | 30 sec | 2.14 × 10⁵ | 1.54 |
| | 1 min | 6.45 × 10⁴ | 2.07 |
| | 5 min | 5.30 × 10² | 4.15 |
| Sample 2 | 30 sec | 9.00 × 10⁴ | 1.92 |
| | 1 min | 9.00 × 10⁴ | 1.92 |
| | 5 min | 1.15 × 10⁴ | 2.81 |
| Sample 3 | 30 sec | 2.15 × 10⁵ | 1.54 |
| | 1 min | 9.65 × 10⁴ | 1.89 |
| | 5 min | 20 | 5.57 |
| E. coli | | | |
| 1.90 × 10⁷ (Time 0) | | | |
| Sample 1 | 30 sec | 2.10 × 10⁵ | 1.96 |
| | 1 min | 4.80 × 10⁴ | 2.60 |
| | 5 min | 5.00 × 10³ | 3.58 |
| Sample 2 | 30 sec | 2.14 × 10⁵ | 2.58 |
| | 1 min | 6.45 × 10⁴ | 2.79 |
| | 5 min | 5.30 × 10² | 3.98 |
| Sample 3 | 30 sec | 2.12 × 10⁵ | 1.95 |
| | 1 min | 9.65 × 10⁴ | 2.29 |
| | 5 min | 7.70 × 10² | 4.39 |

Compositions of the invention kill rapidly (at 100.0% V/V), in general within 5 minutes by approximately 4 logs against Gram −ve and Gram +ve bacteria. This confirms that compositions of the inventions are in the category of an effective antimicrobial agent.

### Example 6—Effectiveness as a Preservative

The ISO 11930 industry standard test was to used measure the effectiveness of the compound to act as a preservative (growth inhibition) for up to 28 days (as tested). Preservative action is used to prevent spoilage and contamination of food, cosmetics, and drugs. First, compositions of the invention were diluted in Glycerol (Tables 7 to 8).

### ISO 11930 Results: Compositions of the Invention Diluted in Glycerol.

TABLE 7A
Concentrations of individual suspensions of S. aureus, E. coli
and P. aeruginosa and concentration of consortium of suspensions.

| Test Organisms & | Dilution and Number of | Bacterial Test |
|---|---|---|
| ATCC/NCTC (Standard) | Colonies | Suspension (N) |
| Staphylococcus aureus | 10⁻⁶: 256; 312 | N: 2.97E+08 |
| ATCC 6538 | 10⁻⁷: 31; 31 | |
| Escherichia coli | 10⁻⁶: >330; >330 | N: 4.45E+08 |
| ATCC 10536 | 10⁻⁷: 49; 40 | |
| Pseudomonas aeruginosa | 10⁻⁶: >330; >330 | N: 3.90E+08 |
| ATCC 15442 | 10⁻⁷: 41; 37 | |
| Bacterial Consortium | 10⁻⁶: >330; >330 | N: 3.35E+08 |
| | 10⁻⁷: 30; 37 | |

TABLE 7B
Effectiveness of Compositions of the Invention (Diluted in Glycerol) as a Preservative
Which Inhibits Growth of S. aureus, E. coli and P. aeruginosa, where Nx is
bacterial consortium test suspension on day x and Rx is log reduction on day x.
Test Procedure at Concentration % (V/V) and Contact Time

| Test | Day | | | Test | Day | | |
|---|---|---|---|---|---|---|---|
| Concentration | 0 | Nₓ | Rₓ | Concentration | 14 | Nₓ | Rₓ |
| 50% | >330; | >3.30E+03 | <3.00 | 50% | 0; | <1.40E+02 | >4.37 |
| | >330 | | | | 0 | | |
| 15% | >330; | >3.30E+03 | <3.00 | 15% | 0; | <1.40E+02 | >4.37 |
| | >330 | | | | 0 | | |
| 5% | >330; | >3.30E+03 | <3.00 | 5% | 0; | <1.40E+02 | >4.37 |
| | >330 | | | | 0 | | |
| 0% | >330; | >3.30E+03 | <3.00 | 0% | 0; | <1.40E+02 | >4.37 |
| | >330 | | | | 0 | | |
| Test | Day | | | Test | Day | | |
| Concentration | 7 | Nₓ | Rₓ | Concentration | 28 | Nₓ | Rₓ |
| 50% | 0; 0 | <1.40E+02 | >4.37 | 50% | 0; 0 | <1.40E+02 | >4.37 |
| 15% | 0; 0 | <1.40E+02 | >4.37 | 15% | 0; 0 | <1.40E+02 | >4.37 |
| 5% | 0; 0 | <1.40E+02 | >4.37 | 5% | 0; 0 | <1.40E+02 | >4.37 |
| 0% | 0; 0 | <1.40E+02 | >4.37 | 0% | 0; 0 | <1.40E+02 | >4.37 |

Preservation Efficacy Criteria A: Rₓ ≥ 3.00 log and no increase in count from previous contact times

TABLE 8A
Concentrations of individual suspensions of C. albicans and
A. brasiliensis and concentration of consortium of suspensions.

| Test Organisms & | Dilution and Number of | Bacterial Test |
|---|---|---|
| ATCC/NCTC (Standard) | Colonies | Suspension (N) |
| Candida albicans | 10⁻⁶: >330; >330 | N: 3.45E+07 |
| ATCC 10231 | 10⁻⁷: 32; 37 | |
| Aspergillus brasiliensis | 10⁻⁶: >165; >165 | N: 2.30E+07 |
| ATCC 16404 | 10⁻⁷: 26; 20 | |
| Bacterial Consortium | 10⁻⁶: >330; >330 | N: 2.55E+07 |
| | 10⁻⁷: 26; 25 | |

TABLE 8B
Effectiveness of Compositions of the Invention (Diluted in Glycerol)
as a Preservative Which Inhibits Growth of C. albicans and A. brasiliensis, where Nx is
bacterial consortium test suspension on day x and Rx is log reduction on day x.
Test Procedure at Concentration % (V/V) and Contact Time

| Test | Day | | | Test | Day | | |
|---|---|---|---|---|---|---|---|
| Concentration | 0 | Nₓ | Rₓ | Concentration | 14 | Nₓ | Rₓ |
| 50% | >330; | >3.30E+03 | <1.88 | 50% | 0; | <1.40E+02 | >3.26 |
| | >330 | | | | 0 | | |
| 15% | >330; | >3.30E+03 | <1.88 | 15% | 0; | <1.40E+02 | >3.26 |
| | >330 | | | | 0 | | |
| 5% | >330; | >3.30E+03 | <1.88 | 5% | 0; | <1.40E+02 | >3.26 |
| | >330 | | | | 0 | | |
| 0% | >330; | >3.30E+03 | <1.88 | 0% | 0; | <1.40E+02 | >3.26 |
| | >330 | | | | 0 | | |
| Test | Day | | | Test | Day | | |
| Concentration | 7 | Nₓ | Rₓ | Concentration | 28 | Nₓ | Rₓ |
| 50% | 0; 0 | <1.40E+02 | >3.26 | 50% | 0; 0 | <1.40E+02 | >3.26 |
| 15% | 0; 0 | <1.40E+02 | >3.26 | 15% | 0; 0 | <1.40E+02 | >3.26 |
| 5% | 0; 0 | <1.40E+02 | >3.26 | 5% | 0; 0 | <1.40E+02 | >3.26 |
| 0% | 0; 0 | <1.40E+02 | >3.26 | 0% | 0; 0 | <1.40E+02 | >3.26 |

Preservation Efficacy Criteria A: Rₓ ≥ 1 and no increase in count from previous contact times

The result of this test (Table 7 to 8) was that compositions of the invention diluted in Glycerol were effective as a preservative against bacteria, yeast and filamentous fungi (mould) (P. aeruginosa, S. aureus, E. coli, C. albicans, A. brasiliensis) up to 28 days (as tested).

ISO 11930 Results: Compositions of the Invention when Diluted in Aqueous Solution at 5.0%; 15% and 50.0%

Next, compositions of the invention were diluted in aqueous solution. As shown in Tables 9 to 10, compositions of the invention are effective as a preservative at 5.0% (no growth observed) against: P. aeruginosa, S. aureus, E. coli, C. albicans, A. brasiliensis.

TABLE 9A
Concentrations of individual suspensions of S. aureus, E. coli
and P. aeruginosa and concentration of consortium of suspensions.

| Test Organisms & | Dilution and Number of | Bacterial Test |
|---|---|---|
| ATCC/NCTC (Standard) | Colonies | Suspension (N) |
| Staphylococcus aureus | 10⁻⁶: 330; 330 | N: 3.35E+08 |
| ATCC 6538 | 10⁻⁷: 37; 30 | |
| Escherichia coli | 10⁻⁶: 299; 280 | N: 2.90E+08 |
| ATCC 10536 | 10⁻⁷: 38; 27 | |
| Pseudomonas aeruginosa | 10⁻⁶: 307; 245 | N: 2.76E+08 |
| ATCC 15442 | 10⁻⁷: 28; 25 | |
| Bacterial Consortium | 10⁻⁶: 240; 240 | N: 1.95E+08 |
| | 10⁻⁷: 21; 18 | |

TABLE 9B
Effectiveness of Compositions of the Invention (Diluted in Aqueous Solution) as a
Preservative Which Inhibits Growth of S. aureus, E. coli and P. aeruginosa, where Nx is
bacterial consortium test suspension on day x and Rx is log reduction on day x.
Test Procedure at Concentration % (V/V) and Contact Time

| Test | Day | | | Test | Day | | |
|---|---|---|---|---|---|---|---|
| Concentration | 0 | Nₓ | Rₓ | Concentration | 14 | Nₓ | Rₓ |
| 50% | >330; | >3.30E+03 | N/A | 50% | 0; | <1.40E+02 | >4.37 |
| | >330 | | | | 0 | | |
| 15% | >330; | >3.30E+03 | N/A | 15% | 0; | <1.40E+02 | >4.37 |
| | >330 | | | | 0 | | |
| 5% | >330; | >3.30E+03 | N/A | 5% | 0; | <1.40E+02 | >4.37 |
| | >330 | | | | 0 | | |
| 0% | >330; | >3.30E+03 | N/A | 0% | >330; | >3.30E+03 | >4.37 |
| | >330 | | | | >330 | | |
| Test | Day | | | Test | Day | | |
| Concentration | 7 | Nₓ | Rₓ | Concentration | 28 | Nₓ | Rₓ |
| 50% | 0; | <1.40E+02 | >4.37 | 50% | 0; | <1.40E+02 | >4.37 |
| | 0 | | | | 0 | | |
| 15% | 0; | <1.40E+02 | >4.37 | 15% | 0; | <1.40E+02 | >4.37 |
| | 0 | | | | 0 | | |
| 5% | 0; | <1.40E+02 | >4.37 | 5% | 0; | <1.40E+02 | >4.37 |
| | 0 | | | | 0 | | |
| 0% | >330; | >3.30E+03 | <2.77 | 0% | >330; | >3.30E+03 | <2.77 |
| | >330 | | | | >330 | | |

Preservation Efficacy Criteria A: Rₓ ≥ 3.00 log and no increase in count from previous contact times.

TABLE 10A
Concentrations of individual suspensions of C. albicans and
A. brasiliensis and concentration of consortium of suspensions.

| Test Organisms & | Dilution and Number of | Bacterial Test |
|---|---|---|
| ATCC/NCTC (Standard) | Colonies | Suspension (N) |
| Candida albicans | 10⁻⁵: >330; >330 | N: 4.95E+07 |
| ATCC 10231 | 10⁻⁶: 54; 45 | |
| Aspergillus brasiliensis | 10⁻⁵: 80; 86 | N: 8.30E+06 |
| ATCC 16404 | 10⁻⁶: 19; 12 | |
| Bacterial Consortium | 10⁻⁵: 208; 205 | N: 2.65E+07 |
| | 10⁻⁶: 18; 35 | |

TABLE 10B
Effectiveness of Compositions of the Invention (Diluted in Aqueous Solution) as a
Preservative Which Inhibits Growth of C. albicans and A. brasiliensis, where Nx is
bacterial consortium test suspension on day x and Rx is log reduction on day x.
Test Procedure at Concentration % (V/V) and Contact Time

| Test | Day | | | Test | Day | | |
|---|---|---|---|---|---|---|---|
| Concentration | 0 | Nₓ | Rₓ | Concentration | 14 | Nₓ | Rₓ |
| 50% | >330; | >3.30E+03 | N/A | 50% | 0; | <1.40E+02 | >3.27 |
| | >330 | | | | 0 | | |
| 15% | >330; | >3.30E+03 | N/A | 15% | 0; | <1.40E+02 | >3.27 |
| | >330 | | | | 0 | | |
| 5% | >330; | >3.30E+03 | N/A | 5% | 0; | <1.40E+02 | >3.27 |
| | >330 | | | | 0 | | |
| 0% | >330; | >3.30E+03 | N/A | 0% | >330; | >3.30E+03 | <1.90 |
| | >330 | | | | >330 | | |
| Test | Day | | | Test | Day | | |
| Concentration | 7 | Nₓ | Rₓ | Concentration | 28 | Nₓ | Rₓ |
| 50% | 0; | <1.40E+02 | >3.27 | 50% | 0; | <1.40E+02 | >3.27 |
| | 0 | | | | 0 | | |
| 15% | 0; | <1.40E+02 | >3.27 | 15% | 0; | <1.40E+02 | >3.27 |
| | 0 | | | | 0 | | |
| 5% | 0; | <1.40E+02 | >3.27 | 5% | 0; | <1.40E+02 | >3.27 |
| | 0 | | | | 0 | | |
| 0% | >330; | >3.30E+03 | <1.90 | 0% | 0; | >3.30E+03 | <1.90 |
| | >330 | | | | 0 | | |

Preservation Efficacy Criteria A: Rₓ ≥ 1 and no increase in count from previous contact times

According to ISO 11930, compositions of the invention produced a log reduction of >4.14 logs at a concentration of 50.0% v/v, 15.0% v/v, and 5.0% v/v as tested after 7 days, 14 days and 28 days at 25° C. against a consortium of Staphylococcus aureus ATCC 6538, Escherichia coli NCTC 10536 and Pseudomonas aeruginosa ATCC 15442 (Table 9). This conforms to Preservation Criteria A for bacteria according to ISO 11930 under the conditions tested.

Also according to ISO 11930, compositions of the invention produced a log reduction of >3.27 logs at a concentration of 50.0% v/v, 15.0% v/v, and 5.0% v/v as tested after 7 days, 14 days and 28 days at 25° C. against a consortium of Candida albicans ATCC 10231 and Aspergillus brasiliensis ATCC 16404 (Table 10).

This conforms to Preservation Criteria A for fungi according to ISO 11930 under the conditions tested.

ISO 11930 Results: Benchmark Control; Glycerol when Diluted in Aqueous Solution at 5.0%; 15% and 50.0%

As a comparison, tests were also performed using Glycerol, instead of Active Glycerol. The results shown below (Tables 11 to 12) confirm that compositions of the invention have beneficial properties above and in addition to any properties of glycerol itself.

### ISO 11930 Results for the Efficacy of Glycerol

TABLE 11A
Concentrations of individual suspensions of S. aureus, E. coli
and P. aeruginosa and concentration of consortium of suspensions.

| Test Organisms & | Dilution and Number of | Bacterial Test |
|---|---|---|
| ATCC/NCTC (Standard) | Colonies | Suspension (N) |
| Staphylococcus aureus | 10⁻⁶: 246; 259 | N: 2.53E+08 |
| ATCC 6538 | 10⁻⁷: 25; 29 | |
| Escherichia coli | 10⁻⁶: >330; >330 | N: 4.05E+08 |
| ATCC 10536 | 10⁻⁷: 44; 37 | |
| Pseudomonas aeruginosa | 10⁻⁶: >330; >330 | N: 4.35E+08 |
| ATCC 15442 | 10⁻⁷: 38; 49 | |
| Bacterial Consortium | 10⁻⁶: 227; 229 | N: 2.30E+08 |
| | 10⁻⁷: 26; 24 | |

TABLE 11B
Effectiveness of Glycerol (Diluted in Aqueous Solution) as a Preservative
Which Inhibits Growth of S. aureus, E. coli and P. aeruginosa, where Nx is
bacterial consortium test suspension on day x and Rx is log reduction on day x.
Test Procedure at Concentration % (V/V) and Contact Time

| Test | Day | | | Test | Day | | |
|---|---|---|---|---|---|---|---|
| Concentration | 0 | Nₓ | Rₓ | Concentration | 14 | Nₓ | Rₓ |
| 50% | >330; | >3.30E+03 | N/A | 50% | 0; | <1.40E+02 | >4.21 |
| | >330 | | | | 0 | | |
| 15% | >330; | >3.30E+03 | N/A | 15% | 122; | 1.20E+03 | 3.28 |
| | >330 | | | | 118 | | |
| 5% | >330; | >3.30E+03 | N/A | 5% | >330; | >3.30E+03 | <2.84 |
| | >330 | | | | >330 | | |
| 0% | >330; | >3.30E+03 | N/A | 0% | >330; | >3.30E+03 | <2.84 |
| | >330 | | | | >330 | | |
| Test | Day | | | Test | Day | | |
| Concentration | 7 | Nₓ | Rₓ | Concentration | 28 | Nₓ | Rₓ |
| 50% | 50; | 4.95E+02 | 3.67 | 50% | 0; | <1.40E+02 | >4.21 |
| | 49 | | | | 0 | | |
| 15% | >330; | >3.30E+03 | <2.84 | 15% | 0; | <1.40E+02 | >4.21 |
| | >330 | | | | 0 | | |
| 5% | >330; | >3.30E+03 | <2.84 | 5% | >330; | >3.30E+03 | <2.84 |
| | >330 | | | | >330 | | |
| 0% | >330; | >3.30E+03 | <2.84 | 0% | >330; | >3.30E+03 | <2.84 |
| | >330 | | | | >330 | | |

Preservation Efficacy Criteria A: Rₓ ≥ 3.00 log and no increase in count from previous contact times

TABLE 12A
Concentrations of individual suspensions of C. albicans and
A. brasiliensis and concentration of consortium of suspensions.

| Test Organisms & | Dilution and Number of | Bacterial Test |
|---|---|---|
| ATCC/NCTC (Standard) | Colonies | Suspension (N) |
| Candida albicans | 10⁻⁵: 256; 289 | N: 2.73E+07 |
| ATCC 10231 | 10⁻⁶: 29; 29 | |
| Aspergillus brasiliensis | 10⁻⁵: >165; >165 | N: 2.10E+07 |
| ATCC 16404 | 10⁻⁶: 22; 20 | |
| Bacterial Consortium | 10⁻⁵: 133; 169 | N: 1.50E+07 |
| | 10⁻⁶: 13; 17 | |

TABLE 12B
Effectiveness of Glycerol (Diluted in Aqueous Solution) as a Preservative
Which Inhibits Growth of C. albicans and A. brasiliensis, where Nx is
bacterial consortium test suspension on day x and Rx is log reduction on day x.
Test Procedure at Concentration % (V/V) and Contact Time

| Test | Day | | | Test | Day | | |
|---|---|---|---|---|---|---|---|
| Concentration | 0 | Nₓ | Rₓ | Concentration | 14 | Nₓ | Rₓ |
| 50% | >330; | >3.30E+03 | N/A | 50% | >330; | >3.30E+03 | <1.65 |
| | >330 | | | | >330 | | |
| 15% | >330; | >3.30E+03 | N/A | 15% | >330; | >3.30E+03 | <1.65 |
| | >330 | | | | >330 | | |
| 5% | >330; | >3.30E+03 | N/A | 5% | >330; | >3.30E+03 | <1.65 |
| | >330 | | | | >330 | | |
| 0% | >330; | >3.30E+03 | N/A | 0% | >330; | >3.30E+03 | <1.65 |
| | >330 | | | | >330 | | |
| Test | Day | | | Test | Day | | |
| Concentration | 7 | Nₓ | Rₓ | Concentration | 28 | Nₓ | Rₓ |
| 50% | >330; | >3.30E+03 | <1.65 | 50% | >330; | >3.30E+03 | <1.65 |
| | >330 | | | | >330 | | |
| 15% | >330; | >3.30E+03 | <1.65 | 15% | >330; | >3.30E+03 | <1.65 |
| | >330 | | | | >330 | | |
| 5% | >330; | >3.30E+03 | <1.65 | 5% | >330; | >3.30E+03 | <1.65 |
| | >330 | | | | >330 | | |
| 0% | >330; | >3.30E+03 | <1.65 | 0% | >330; | >3.30E+03 | <1.65 |
| | >330 | | | | >330 | | |

Preservation Efficacy Criteria A: Rₓ ≥ 1 and no increase in count from previous contact times

According to ISO 11930, Glycerol produced a log reduction of 3.67 logs at a concentration of 50.0% v/v as tested after 7 days, which increased to >4.21 logs at 14 days and 28 days at 25° C. against a consortium of Staphylococcus aureus ATCC 6538, Escherichia coli NCTC 10536 and Pseudomonas aeruginosa ATCC 15442 (Table 11). This conforms to Preservation Criteria A for bacteria according to ISO 11930 under the conditions tested.

According to ISO 11930, Glycerol did not produce a measurable log reduction at concentrations of 50.0% v/v, 15.0% v/v, 5.0% v/v and 0.0% v/v as tested after 7 days, 14 days and 28 days at 25° C. against a consortium of Candida albicans ATCC 10231 and Aspergillus brasiliensis ATCC 16404 (Table 12). This does not conform to Preservation Criteria A for fungi according to ISO 11930 under the conditions tested. These results show that Compositions of the invention are more effective as a preservative at least 1/10 the concentration of Glycerol and are in addition to being effective against bacteria, an effective preservative against yeast and fungi at this concentration.

Glycerol is a preservative, but at levels>50.0% V/V for bacteria and >60.0% for fungi (not tested, shows a fail at 50.0%). At this concentration glycerol is “sticky” and can be a skin desiccant.

Glycerol is normally used in cosmetic products as a 2-5% emulsion. The above results for compositions of the invention diluted in aqueous solution at 5.0% V/V indicate that compositions of the invention are a preservative at the normal range of concentration it is used for its physical properties in cosmetics.

Compositions of the invention can potentially have a dual function in products, firstly to be used for its physical properties, and secondly as a (natural) preservative. This can have significant economic consequence for the cost of manufacture of preserved products.

### Example 7—Virucidal Activity

Virucidal assays were carried out according to industry standards, Modified BS EN 14476 2013+A2 2019 Screen, a quantitative suspension test for the evaluation of virucidal activity.

Modified BS EN 14476 2013+A2 2019 SCREEN Results: Compositions of the Invention when Diluted in Aqueous Solution at 80.0% V/V.

TABLE 13
Modified EN14476 Virus Screen Suspension Test for
the Efficacy of Active Glycerol, Batch DOM-05-12-
22, BT-AGP-04 from AGP Innovation Ltd Against Vaccinia
Virus VR-1549 Under CLEAN Conditions

| | | | |
|---|---|---|---|
| Exposure Time / min | TCID₅₀ / mL | Log(TCID₅₀) | Log Reduction |

Composition of Invention (80.0% V/V)

| | | | |
|---|---|---|---|
| 0 | 3.16E+06 | 6.50 | 4.00 |
| 5 | 3.16E+02 | 2.50 | |

Virus Control

| | | | |
|---|---|---|---|
| 0 | 1.00E+06 | 6.00 | N/A |
| 5 | 4.64E+06 | 6.67 | |

Compositions of the invention achieved a 4.0 log reduction at a concentration of 80.0% v/v as tested after 5 minutes at 20° C. against Vaccinia virus VR-1549 Elstree strain/Vero cells (Table 13).

Compositions of the invention can be defined as virucidal (>4.0 log₁₀ reduction in 5 minutes) against enveloped viruses.

Compositions of the invention were not tested for virucidal activity at lower concentrations or shorter contact times against enveloped viruses, and therefore may be effective at lower concentrations.

### CONCLUSIONS

Compositions of the inventions have value as a natural microbial growth inhibitor of bacteria, yeast and filamentous fungi (mould) at concentrations significantly lower than Glycerol. This enables such compositions to be an effective preservative in food, cosmetic and drugs as well as a source of new potential applications as an antimicrobial. The potent activity of killing bacteria and yeast (biocidal activity) by compositions of the invention is a significant activity that is unique for compositions of the invention compared to Glycerol and has significant implications for the use and applications of such compositions.

The mode of action of growth inhibition and biocidal activity of compositions of the invention is of relevance to combating antimicrobial resistance (AMR). AMR will be responsible for more human mortality than all cancer-related deaths by 2050.

The results indicate that compositions of the invention have significant additional antimicrobial activities to compared Glycerol. These can confer additional/synergistic/complementary activity compared to Glycerol-which is a very widely used ingredient in food, cosmetic and drug products. The ability of compositions of the invention to replace Glycerol in products (whilst retaining, for example, the physical properties of Glycerol) is advantageous.

### Example 8—Production of Active Compositions Without Catalyst

Compositions of the invention with anti-microbial activity were prepared without using a catalyst.

The NPA assay was used, as previously in Example 3.

TABLE 14
NPA Assay Results For Compositions of the Invention
Without Catalyst, Two Days After Mixing Reagents

| | | Mean Result / | | |
|---|---|---|---|---|
| Sample | | % w/v phenol | DHA | MGO |
| No. | Sample Details | equivalent | (mg/kg) | (mg/kg) |
| 93 | Glycerol + Water + | 18.5 | 1134 | <50 |
| | H₂O₂ | | | |
| 94 | Glycerol + Water + | 17.5 | 1117 | <50 |
| | H₂O₂ | | | |
| 95 | Glycerol + Water + | 17.0 | 1125 | <50 |
| | H₂O₂ | | | |
| 96 | Glycerol + Water + | 21.0 | 1723 | <50 |
| | H₂O₂ + DHA* | | | |

*Spiked at 2000 mg/kg

### Observations and Postulations

DHA is synthesised when the catalyst is omitted from the formulation, generally at a lower concentration compared with the compositions obtained when a catalyst is included (for example about ⅓ the level). The NPA test results confirmed that an NPA antimicrobial activity of around 18.0-20% phenol equiv. is produced over the 2-day reaction/incubation period in the absence of a catalyst (Table 14). It is confirmed that no significant MGO is produced in the absence of the catalyst. Addition of DHA to the initial formulation at mixing appears to either inhibit further DHA formation, and/or accelerate the reaction of DHA to other molecules (possibly glyceraldehyde initially). It appears that the use of a catalyst (preferably ferrous lactate) is essential for the synthesis of significant levels of methyl glyoxal over such a 2-day production process. It appears that dihydroxyacetone (DHA) is formed over this 2-day production process when the catalyst is omitted, albeit at lower concentrations compared with compositions produced using a catalyst. Levels are around one third of those produced when the catalyst is present.

A non-peroxide activity (NPA) of around 18.0 to 20.0% phenol equiv. is produced in the composition of the invention over the 2-day production process when the catalyst is omitted. This antimicrobial activity is thought to result from the synthesis of various organic acids also noted in the AG product formulated with catalyst. Compositions of the invention produced without a catalyst have antimicrobial activity. The methods performed without a catalyst, and the resulting compositions of the invention, are advantageous in several ways.

TABLE 15
pH of the Compositions at 0 to 72 hours

| Sample | | | | | | | | |
|---|---|---|---|---|---|---|---|---|
| No. | Sample Details | 0 h | 0.5 h | 1.5 h | 3 h | 5 h | 24 h | 72 h |
| 14 | Glycerol + Water + Water | 5.0 | 5.0 | 5.0 | 4.8 | 4.8 | 5.0 | 4.9 |
| 15 | Glycerol + Catalyst + H₂O₂ | 4.1 | 4.1 | 3.9 | 3.1 | 3.0 | 2.9 | 2.9 |
| 16 | Glycerol + Water + H₂O₂ | 5.0 | 5.2 | 5.0 | 4.7 | 4.2 | 3.4 | 3.1 |
| 17 | Glycerol + Catalyst + Water | 5.0 | 5.0 | 4.9 | 4.6 | 4.5 | 4.4 | 4.1 |

The first 30 minutes were held at ambient temperature, followed by being held at 37° C. for the remainder of the 72 hours.

There was a ‘rapid’ pH drop on mixing of reagents for the formulation with catalyst, glycerol and hydrogen peroxide (Table 15).

There was an apparent slower decline in pH with no catalyst—this supports the understanding that H₂O₂ reacts with glycerol to form the various acids noted as being present in the formulation when the catalyst is absent.

The final pH of the compositions formed with and without the use of a catalyst is very similar.

The compositions comprising a catalyst produced a rapid pH drop. Sample 17 results suggest that the catalyst alone is not responsible for the drop in pH as observed, as this is considered to result from the rapid synthesis of various acids.

It is considered unlikely that MGO contributes to the acidity as the final pH of samples 15 and 16 is similar. This suggests a similar composition of acids in both samples, or composition of acids both yielding similar pHs.

TABLE 16
pH of the Compositions at 30 mins, 48 hours and 72 hours

| Sample | | | | |
|---|---|---|---|---|
| No. | Sample Details | 30 mins | 48 hrs | 72 hrs |
| 93 | Glycerol + Water + H₂O₂ | 5.0 | 3.3 | 3.1 |
| 94 | Glycerol + Water + H₂O₂ | 5.0 | 3.3 | 3.1 |
| 95 | Glycerol + Water + H₂O₂ | 5.0 | 3.3 | 3.1 |
| 96 | Glycerol + Water + H₂O₂ + | 4.9 | 3.2 | 3.1 |
| | DHA* | | | |

*Spiked at 2000 mg/kg

These results (Table 16) provide confirmation of the noted slower decline in pH with no catalyst. This supports the understanding that H₂O₂ reacts with glycerol to form the various acids noted as being present in the formulations of the invention both in the presence or absence of a catalyst. Presence of a catalyst appears to affect the rate of pH reduction but not the final pH attained. Final pH values attained are similar to those obtained in the presence of a catalyst. Addition of DHA to the initial formulation has minimal/no significant effect on either the rate of pH decline (acid formation) or pH values attained.

The above embodiments are to be understood as illustrative examples of the invention. Further embodiments of the invention are envisaged. It is to be understood that any feature described in relation to any one embodiment may be used alone, or in combination with other features described, and may also be used in combination with one or more features of any other of the embodiments, or any combination of any other of the embodiments. Furthermore, equivalents and modifications not described above may also be employed without departing from the scope of the invention, which is defined in the accompanying claims.

1. An antimicrobial composition comprising glycerol, dihydroxyacetone and methylglyoxal.

2. The antimicrobial composition of claim 1, further comprising one or more compounds selected from: glycolic acid, formic acid, acetic acid, 2-propanol, acetone, glyceraldehyde and acrolein.

3. The antimicrobial composition of claim 1, wherein the dihydroxyacetone content is between about 150 mg kg⁻¹ and 4000 mg kg⁻¹.

4. The antimicrobial composition of claim 1, wherein the methylglyoxal content is between about 50 mg kg⁻¹ and 1200 mg kg⁻¹.

5. The antimicrobial composition of claim 1, wherein the pH of the composition is lower than about 4.0.

6. The antimicrobial composition of claim 1, wherein the composition has at least one of the following properties: antibacterial, antifungal, antiviral and biocidal.

7. The antimicrobial composition of claim 1, wherein the composition has a non-peroxide activity against Staphylococcus aureus of more than 15% w/v phenol equivalent.

8. The antimicrobial composition of claim 1, wherein the composition is antibacterial, characterised by a minimum inhibitory concentration of 10% or less against bacteria.

9. The antimicrobial composition of claim 1, wherein the composition is antifungal, characterised by a minimum inhibitory concentration of 10% or less against fungi.

10. The antimicrobial composition of claim 1 which is food grade.

11. A method of making the antimicrobial composition of claim 1, comprising mixing: a reagent comprising glycerol and an oxidant; optionally with a catalyst.

12. The method of claim 11, wherein the oxidant comprises hydrogen peroxide.

13. The method of claim 11, wherein the catalyst comprises ferrous (II) lactate.

14. A product comprising the antimicrobial composition as defined in claim 1 for use to prevent the growth of, or kill, at least one of:
bacteria, yeasts, fungi and viruses.

15. Use of the antimicrobial composition as defined in claim 1 in the treatment of a wound, wherein a product comprising the composition is applied to the wound.

16. A wound dressing comprising the antimicrobial composition as defined in claim 1.

17. A food product comprising the antimicrobial composition as defined in claim 1.

18. A cosmetic product comprising the composition as defined in claim 1.

19. A cleaning product comprising the composition as defined in claim 1.

20. Use of the antimicrobial composition as defined in claim 1 in the prevention or treatment of a skin condition.

21. (canceled)
